(19) United States
(12) Patent Application Publication
(10) Pub. No.: US 2020/0163746 A1
(43) Pub. Date: May 28, 2020

(54) CANNABINOID-INFUSED POST-OPERATIVE DENTAL DRESSING

(71) Applicants: David M. Tumey, Coral Springs, FL (US); Harry Panahi, Coral Springs, FL (US)

(72) Inventors: David M. Tumey, Coral Springs, FL (US); Harry Panahi, Coral Springs, FL (US)

(21) Appl. No.: 16/675,325

(22) Filed: Nov. 6, 2019

Related U.S. Application Data
(60) Provisional application No. 62/771,318, filed on Nov. 26, 2018.

(51) Int. Cl. A61C 19/06; A61F 7/12; A61F 7/02; A61K 9/00; A61K 9/70; A61K 31/05; A61L 15/44; A61L 15/42; A61K 36/82; A61K 36/9066

(52) U.S. Cl. CPC A61C 19/063; A61F 7/12; A61F 7/02; A61K 9/0063; A61K 9/009; A61F 2007/0017; A61K 31/05; A61L 15/44; A61L 15/425; A61K 36/82; A61K 36/9066; A61K 9/7007

(57) ABSTRACT

A dental dressing to promote hemostasis, reduce inflammation, alleviate pain, and lower incidence of infection associated with dental surgical procedures. The dressing comprises a three-dimensional absorbent matrix to assist in the removal of exudate associated with iatrogenic trauma, and embedded cannabinoid-infused therapeutic phytoconstituents to provide substantially localized therapy to the surgical site. The cannabinoid-infused therapeutic phytoconstituents are substantially contained within a porous dressing pouch shapeable to substantially engage the iatrogenic trauma while fitting generally over and about the surgical site. At least part of the dressing matrix substantially conforms to the surgical site, thereby engaging the therapeutic phytoconstituents intimately with the iatrogenic trauma.

### FIELD OF THE INVENTION

The present general inventive concept relates to a cannabis-infused wound dressing for dental surgical procedures that can be applied during or after a dental procedure or trauma to promote hemostasis, reduce inflammation, alleviate pain, and lower incidence of infection.

### BACKGROUND OF THE INVENTION

Within the human oral cavity, teeth are disposed in dental alveoli, commonly referred to as sockets. Dental alveoli and teeth are incorporated on both the maxilla (upper jaw) and mandible (lower jaw). Normally, twenty (20) primary or “baby” teeth are originally grown that eventually fall out and are subsequently replaced by thirty-two (32) permanent or “adult” teeth which include the four (4) third molars or “wisdom teeth”. It is noted, however, many individuals may not grow all of the third molars and some will not grow any of the third molars.

Various problems can manifest in the oral cavity involving the mucosa, gums, teeth, tooth sockets and bone. For example, bacteria can be a primary cause of tooth decay. Sometimes, the extent of this decay necessitates the extraction or removal of the tooth by a dentist, oral surgeon, or other dental practitioner. Tooth extraction can be accomplished, for example, by grasping the tooth with forceps and performing a rocking motion to loosen the tooth from the alveolar bone structure. The tooth is loosened from the alveolar bone when connecting ligaments, or periodontal ligaments, that secure the tooth in place are progressively broken. Eventually, the tooth can be removed from the socket, thereby exposing the dental alveolus. Similarly, dental elevators can be employed to loosen and elevate teeth in their sockets prior to extraction to help prevent damage to adjacent anatomical structures. Elevators are utilized to cut periodontal ligaments and free tissue from bone.

Tooth extraction may also be required when, for instance, specific teeth are causing overcrowding in the mouth, malocclusion, or preventing the eruption of additional teeth from the gum line. Likewise, prophylactic wisdom tooth extraction has become more popular over the years. Further, tooth extraction frequently occurs prior to and in preparation for orthodontic treatment such as braces or dentures. Additionally, tooth extraction may also be necessitated by gum disease, and/or neoplasia.

Trauma to the oral cavity and surrounding tissue and structures can also bring about or require the removal of teeth. Occasionally, self-induced trauma is caused by complications with the extraction of primary teeth. More frequently, car accidents, sporting injuries, and the like cause trauma to the oral cavity and result in unintentional tooth extraction.

Pain, inflammation and bleeding are common during and after most dental surgical procedures and traumatic incidents. Pain is often caused by alveolitis, or swelling of the tooth socket, whereas bleeding can result from the manipulation and/or destruction of proximate soft tissue. Alveolar osteitis, or dry sockets, can also emerge in the exposed tooth socket following tooth extraction after a blood clot forms, thus depriving the tooth socket of blood. While the cause of this particular complication is substantially unknown, the concomitant pain is immediately appreciated by anyone who has experienced dry sockets. In this regard, preventive measures are generally encouraged to keep the exposed tooth socket clean and moist. Furthermore, treatment is usually effectuated by applying medications containing, for example, eugenol, benzocaine, and/or iodophorm. Hydrogel dressings, which release water to maintain a moist wound environment, have also been used in this circumstance.

Methods of facilitating healing, alleviating pain and inflammation, and ameliorating bleeding are known in the art. Cotton gauze pads, tulle, cottons swabs, or the like can be inserted into the oral cavity and compressed against the exposed iatrogenic trauma to promote hemostasis and absorb blood, third-space fluids and other exudates. Pressure is particularly important for achieving hemostasis; however bioactive ingredients such as chitosan can serve as an effective ameliorator. For example, U.S. Pat. No. 7,897,832 discloses dental dressings that are fabricated from hydrophilic polymer sponge structures, such as densified-chitosan biomaterial.

Another well known agent that can be combined with pressure to assist in achieving hemostasis is tannin. Tannins are present in many woods, where they provide a partial defense against wildfires, decomposition, and infestation. Tannin is also a frequent ingredient in tea and is typically sold commercially as tannic acid. Tannin typically operates as a vasoconstrictor in tissue and is normally topically applied. Therefore, doctors frequently suggest biting down on a moist tea bag containing tannin following tooth extraction or other surgical procedures to help inhibit bleeding. Research also suggests that tannin has antiviral, antimicrobial, and antibacterial properties as well.

Hemostasis is generally achieved, in part, by coagulation, or clotting, of the blood. In brief, coagulation occurs when damaged leaky blood vessels are covered by platelets and fibrin. Fifteen primary factors are required for blood to begin the coagulation process. Among those factors are fibrinogen and calcium. Both the intrinsic and extrinsic pathways associated with coagulation require copious amounts of calcium to initiate and progress the cascade of biological reactions that cause coagulation. Chitosan is also known to help facilitate coagulation.

Pain and inflammation associated with dental surgical procedures are often addressed by applying cold therapy to the oral cavity. Typically, this is implemented by placing an ice pack or similar arrangement on the exterior of the cheek, proximate the exposed surgical site. Most individuals experience some pain relief when their wound site is exposed to cold therapy. Cold therapy is an effective pain remedy because cold temperature, when applied to nerve cells, lowers the baseline/resting membrane potential of the individual neurons. Neurons transmit electrical signals in response to stimuli by increasing the voltage within their membranes to surpass a trigger threshold level, also referred to as action potential. In order to achieve action potential for the bio-electric signal to be transmitted to the next neuron, the voltage increase from the resting membrane potential of approximately −70 millivolts must exceed a trigger threshold level. Thus, when the resting membrane potential is lowered, for instance by cold therapy, a higher voltage increase will be required to achieve the threshold trigger level required for transmission of the neural signal. Therefore, lowering the resting membrane potential enough so as to prevent the voltage increase from surpassing the trigger threshold level will help prevent the sensation of pain to be transmitted between neurons and ultimately the brain.

Likewise, inflammation often subsides when cold therapy is applied to the inflamed tissue. Inflammation can result from an excess quantity of blood and fluid leaking from ruptured capillaries. Reduced temperature causes the blood vessels within the swollen tissue to constrict or narrow. This vasoconstriction takes place when the smooth muscle within the blood vessels contract, thus reducing and/or preventing further leakage of blood and serum. By this same principle, vasoconstriction also plays an important role in achieving hemostasis.

Ice packs, however, often go too far in terms of applying cold therapy to the exterior of the oral cavity. Ice and other frozen objects can create cold burns on the skin. Even greater danger manifests when ice causes the skin to become numb, thereby preventing the patient from noticing the tissue damage taking place. Further, cold therapy applied on the exterior of the cheek is not a very efficient method of application of such therapy to an exposed tooth socket, for example. The temperature gradient between the exterior of the cheek, the skin and tissue, and exposed tooth socket prevents the efficient application of localized cold therapy directly at the site of the trauma. In addition, the regional application of cold therapy diminishes the pain relief desired for the specific wound site. Put differently, the lack of focused cold therapy applied locally to the iatrogenic trauma sacrifices some of the therapeutic benefits of the cold therapy.

Therefore, what is desired is a dental dressing capable of applying localized therapy directly to a surgical site. More particularly, what is desired is a dental dressing containing a malleable therapeutic matrix that can fit substantially over a surgical site in intimate contact with an iatrogenic trauma, and apply localized therapy to the iatrogenic trauma while also helping to prevent post-operative complications. Further desired is a dental dressing capable of including topical active ingredients which, when applied to an iatrogenic trauma, promote hemostasis, healing and reduced pain and inflammation.

### SUMMARY OF THE INVENTION

The present general inventive concept provides a dental dressing to promote hemostasis, reduce inflammation, alleviate pain, and lower incidence of infection associated with dental surgical procedures. In some embodiments, the dental dressing comprises a three-dimensional absorbent matrix to assist in the removal of exudate associated with iatrogenic trauma, and embedded cannabinoid-infused therapeutic phytoconstituents to provide substantially localized therapy to the surgical site. The cannabinoid-infused therapeutic phytoconstituents are substantially contained within a porous dressing pouch shapeable to substantially engage the iatrogenic trauma while fitting generally over and about the surgical site. At least part of the dressing matrix substantially conforms to the surgical site, thereby engaging the therapeutic phytoconstituents intimately with the iatrogenic trauma.

In some embodiments of the present general inventive concept, the dressing pouch is filter-paper, muslin, silk, cotton gauze, tulle, cotton, and/or any combination thereof. In other embodiments, the dressing pouch includes hydrogel, alginate, hydrofibre, chitosan, and/or any combination thereof.

In some embodiments, the therapeutic phytoconstituents are substantially made of a mixture of tea blends, turmeric and cannabinoids substantially contained within the porous dressing pouch. In some embodiments, the mixture is substantially non-migrating. In other embodiments, the therapeutic phytoconstituents are substantially made of tea blends, turmeric and cannabinoids substantially contained within a generally rectangular porous dressing pouch. In yet other embodiments, the therapeutic phytoconstituents are substantially made of tea blends, turmeric and cannabinoids substantially contained within a generally round porous dressing pouch.

In some embodiments of the present general inventive concept, the porous dressing pouch is substantially U-shaped so as to engage one or more exposed alveoli. In other embodiments, the porous dressing pouch includes a substantially U-shaped cross section, and is elongated to engage a plurality of adjacent, exposed alveoli. In other embodiments, the porous dressing pouch is substantially H-shaped so as to engage substantially aligned maxillary and mandibular exposed alveoli. In yet other embodiments, the porous dressing pouch includes a substantially H-shaped cross section and is elongated to engage a plurality of adjacent, substantially aligned, exposed maxillary and mandibular alveoli.

In some embodiments, the dental dressing further includes a moisture-liberating element, with the element embedded and contained substantially within the dressing pouch. In some embodiments, the moisture-liberating element includes hydrogel, hydrocolloid, hydrofibre, and/or any combination thereof. In some embodiments, the dental dressing includes a topical agent element, with the element embedded and contained substantially within the dressing pouch. In some embodiments, the topical agent element includes clove oil (eugenol) infused with cannabidiol. In other embodiments, the topical agent element includes benzocaine, iodophorm, soluble aspirin, zinc oxide, and/or any combination thereof.

Additional aspects and advantages of the present general inventive concept will be set forth in part in the description which follows, and, in part, will be obvious from the description, or may be learned by practice of the present general inventive concept.

### BRIEF DESCRIPTION OF THE DRAWINGS

The following example embodiments are representative of exemplar techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 is a perspective view of an example embodiment of the present general inventive concept, showing an exterior aspect of an exemplar dressing pouch;

FIG. 2 is a side view of the embodiment shown by FIG. 1;

FIGS. 3A & 3B are cross-sectional views of the embodiment shown in FIG. 2, with FIG. 3A illustrating an example embodiment of the present inventive concept and FIG. 3B portraying another example embodiment of the present inventive concept;

FIG. 4 is an exploded view of an example embodiment of the present inventive concept engaging an exposed tooth socket;

FIG. 5 depicts the embodiment shown in FIG. 4 substantially ensconced within an exposed tooth socket;

FIG. 6 is a more detailed view of the example embodiment portrayed in FIG. 5;

FIG. 7 is a cross-sectional view of the example embodiment of FIG. 6;

FIGS. 8A & 8B are cross-sectional views of two example embodiments of the present inventive concept, both with a cannabinoid-infused phytotherapeutic matrix and an embedded moisture-liberating element contained substantially within the porous dressing pouch;

FIGS. 9A & 9B are cross-sectional views of two example embodiments of the present inventive concept, both with a cannabinoid-infused phytotherapeutic matrix, an embedded moisture-liberating element, and topical agent element contained substantially within the porous dressing pouch;

FIGS. 10A & 10B are cross-sectional views of two example embodiments of the present inventive concept that can be applied to substantially aligned maxillary and mandibular exposed tooth sockets, both with a cannabinoid-infused phytotherapeutic matrix, an embedded moisture-liberating element, and topical agent element contained substantially within the porous dressing pouch;

FIGS. 10C & 10D are cross-sectional views of two example embodiments of the present inventive concept engaging substantially aligned, mandibular and maxillary exposed tooth sockets, with FIG. 10C including a cannabinoid-infused phytotherapeutic matrix contained substantially within a porous dressing pouch, and FIG. 10D including a cannabinoid-infused phytotherapeutic matrix, an embedded moisture-liberating element, and topical agent element contained substantially within a porous dressing pouch.

FIG. 11 depicts an example embodiment of the present inventive concept engaging substantially aligned maxillary and mandibular exposed wisdom tooth sockets;

FIG. 12 depicts an example embodiment of the present inventive concept engaging adjacent, exposed mandibular tooth sockets;

FIG. 13 depicts an example embodiment of the present inventive concept engaging a plurality of adjacent, substantially aligned, maxillary and mandibular exposed tooth sockets;

FIG. 14 depicts an example embodiment of the present inventive concept engaging an entire mandibular row of exposed tooth sockets.

### DETAILED DESCRIPTION OF THE PREFERRED EMBODIMENT

Reference will now be made to various example embodiments of the present inventive concept, examples of which are illustrated in the accompanying drawings and illustrations. The exemplar embodiments are described herein in order to explain the present general inventive concept by referring to the figures. The following detailed description is provided to clarify the methods, apparatuses, and/or systems described herein. Accordingly, various changes, modifications, and equivalents of the methods, apparatuses, and/or systems described herein will be apparent to those of ordinary skill in the art.

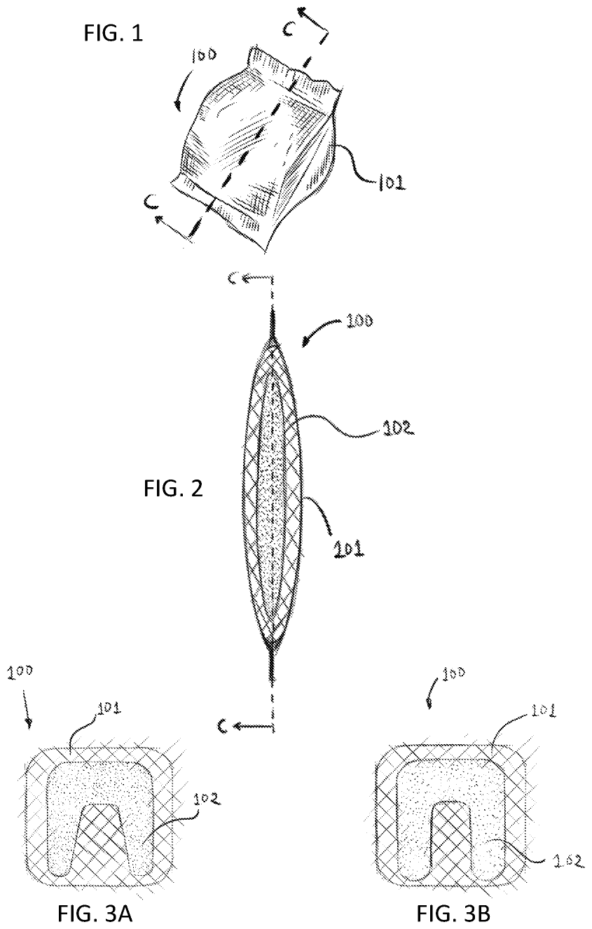

A dental wound dressing 100 for dental surgical procedures, in accordance with various embodiments of the present general inventive concept, is shown generally in FIGS. 1-3B collectively. FIG. 1 depicts an exterior, perspective view of an example embodiment of the present general inventive concept. FIG. 2 depicts the same embodiment from FIG. 1, only from a side view with a cross-sectional cutting line “C”, designating a cross sectional view of the exemplar dressing shown in FIG. 1 as well as representative cross sectional views of alternative embodiments shown in FIGS. 3, 8, 9 & 10.

A dental dressing 100 for treating dental surgical procedures includes an exterior dressing pouch 101. The dressing pouch 101 can be a comprised of dressing material that adapts to various spatial requirements. For instance, one suitable dressing pouch 101 material is cotton gauze. Cotton gauze can be shaped, configured, and adjusted to conform to a given space. Moreover, cotton gauze is readily adaptable to the particular morphology and topology concerns of an exposed tooth socket, for example. Also important, gauze provides an appropriate medium to absorb blood and other exudate from the wound site. One skilled in the art will understand that the present general inventive concept is, of course, not limited to using gauze. For example, filter-paper, muslin, silk, cotton swabs, tulle, and the like, or any combination thereof, can also be utilized to achieve excellent clinical efficacy. In addition, dressing pouches containing topical agents and bio-ingredients such as, for example, hydrogel, alginate, hydrofibre, collagen, chitosan, or any combination thereof, can also be utilized. Other suitable dressing pouches will be apparent to those skilled in the art, and may be used without deviating from the scope or spirit of the present general inventive concept.

Contained substantially within the dressing pouch 101, is a cannabinoid-infused phytotherapeutic matrix 102. In the illustrated embodiment, the phytotherapeutic matrix 102 is substantially comprised of tea blends, turmeric and cannabinoids. More specifically, in the illustrated embodiment, the phytotherapeutic matrix 102 is substantially comprised of a substantially non-migrating mixture. Otherwise stated, the phytotherapeutic matrix 102 in the present embodiment is semi-rigid and effective at substantially retaining its shape under pressure.

Additionally, in some embodiments of the general present inventive concept, the phytotherapeutic matrix 102 can include a thermally active gel-like substance. Otherwise stated, the matrix 102 can substantially retain its temperature after introduction into hot or cold environments and subsequent removal therefrom. For example, the dressing can be placed in a refrigerator or freezer until cooled to a desired temperature. Upon removal from the cold environment, the dressing will substantially retain its cold temperature and subsequently provide cooling of the surgical site when placed within the oral cavity.

One skilled in the art will understand that the present general inventive concept is not limited to a phytotherapeutic matrix 102 that includes gel. On the contrary, other substances with similar properties can comprise the phytotherapeutic matrix 102. For instance, ammonium nitrate and water contained within a common housing member can combine to produce an endothermic reaction resulting in a cold product. Similarly, cannabinoid-infused clove oil can also be mixed into the matrix and stored in a cold environment. Of course, in order to create a phytotherapeutic matrix 102, the above-mentioned ingredients will have to be contained in a porous dressing pouch that can be readily inserted into an oral cavity and engage a surgical site, while also substantially fitting between contiguous teeth. Further, in some embodiments, the porous dressing pouch can be relatively pliable, so as to prevent damage to teeth exerting pressure on the dental dressing 100. One skilled in the art will recognize that several polymer-based materials can also be utilized to create the porous dressing pouch that is adaptable for insertion into an oral cavity. One skilled in the art will also understand, however, that the present general inventive concept is not limited to the above-mentioned substances. Other substances capable of retaining a cold temperature and being contained within a porous dressing pouch can also be used without departing from the scope or spirit of the present general inventive concept.

FIG. 3A depicts a cross-sectional view of an example embodiment of the present general inventive concept with the phytotherapeutic matrix 102 in the general shape of a “U,” but having tapered and angled side arms. Otherwise stated, the embodiment illustrated in FIG. 3A includes a cover portion to cover an exposed tooth socket, for example, and two tapered arm members angularly extending from opposing sides of the socket cover segments. FIG. 3B depicts a cross-sectional view of another example embodiment of the present general inventive concept with the phytotherapeutic matrix 102 in the general shape of a “U” without incorporating tapered or angled arm members. Otherwise stated, the embodiment in FIG. 3B includes a socket cover portion and two non-tapered arm members perpendicularly extending from the cover portion.

When engaging an exposed tooth socket, the phytotherapeutic matrix 102 can be oriented in such a way that it substantially covers and surrounds the exposed tooth socket while also fitting between contiguous teeth. More specifically, when engaging an exposed tooth socket, the cover portions of the present example embodiments can sit on top of, or be disposed directly beneath, one or more exposed tooth sockets, while the two tapered arm members laterally flank one or more exposed tooth sockets on opposing sides of the oral epithelium. Otherwise stated, the phytotherapeutic matrix 102 can superpose the exposed tooth socket as well as engage both sides of the oral epithelium and gingival tissue proximate the exposed socket, with one arm member engaging the facial surface of the proximate gum tissue, and the other arm member engaging the lingual surface of the proximate gum tissue.

The primary difference between the embodiment illustrated in FIG. 3A and the one illustrated in FIG. 3B is shape. The angled and flared arm members of the therapeutic matrix in FIG. 3A can provide a better fit for individuals with larger-sized gums. Depending on the flexibility/rigidity of the matrix and pouch comprising the dressing in the illustrated embodiment, the overall shape of the phytotherapeutic matrix 102 can remain substantially the same even with force exerted by the opposite jaw. Otherwise stated, in some embodiments the phytotherapeutic matrix 102 can be semi-rigid so as to substantially prevent significant flexing in the shape of the phytotherapeutic matrix 102. Thus, for individuals with larger gums, the angled and tapered arm members can facilitate an improved fit, while still permitting the dressing to remain substantially between contiguous teeth. Further, the tapered arm members can also permit the inclusion of additional absorbent material. Otherwise stated, tapered arm members occupy less volume than the non-tapered arm members of FIG. 3B, and therefore permit the absorbent material to occupy the excess volume that would be otherwise be occupied by the non-tapered arm members. Providing additional absorbent material to the dental dressing 100 will allow the dental dressing 100 to generally absorb more exudate from the wound site. While the foregoing description is generally directed specifically to an exposed tooth socket, it should be understood by anyone of ordinary skill in the art that the exemplar dental dressing 100 described in the previous paragraphs is suitable for use over any iatrogenic trauma or surgical site and is not in any way limited to exposed tooth sockets.

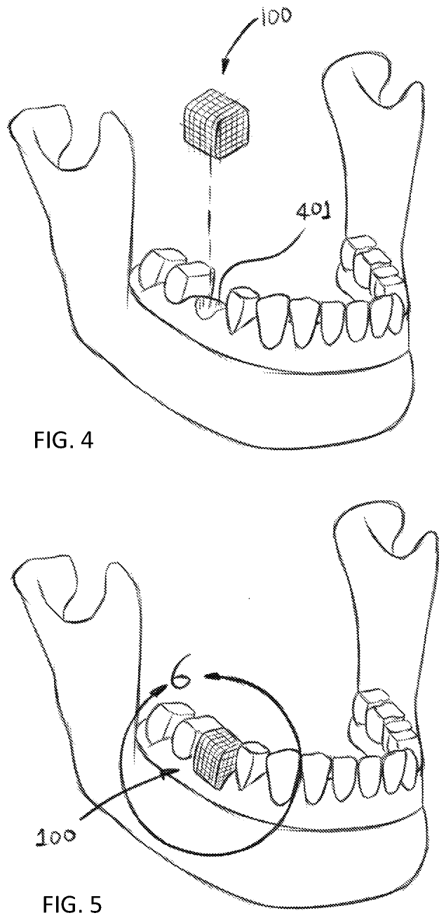

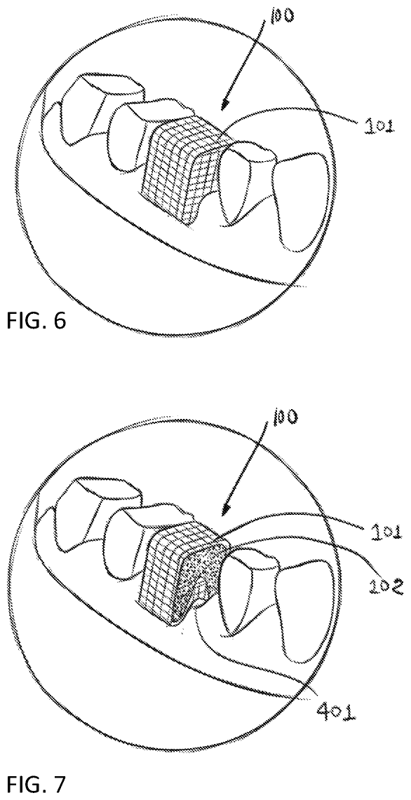

FIGS. 4-7 depict an example embodiment of the present general inventive concept engaging an exposed tooth socket. FIG. 4 depicts an exploded view of an example dental dressing 100 engaging an exposed tooth socket 401. FIGS. 5-7 depict an example dental dressing 100 ensconced within an exposed tooth socket 401. Specifically, FIG. 6 depicts a more detailed view of the embodiment portrayed in FIG. 5, and FIG. 7 depicts a cross-sectional view of the embodiment portrayed in FIG. 6.

Each exposed tooth socket 401, and for that matter any iatrogenic trauma, contains particular morphological and topological concerns. For example, an exposed tooth socket 401 generally contains a cavity, into which the root of a tooth is generally disposed and secured to the alveolar (jaw) bone with periodontal ligaments. Further, the gingival tissue immediately abutting and surrounding the cavity has distinct contours and shapes. For instance, the marginal and attached gingiva contain depressions, grooves, and indentations. In this regard, the dressing 100 can substantially conform to the topology and morphology of the exposed tooth socket 401 and proximate gingival and epithelial tissue, and can be compressed to apply appropriate pressure for promoting hemostasis. The dressing pouch 101 can also be compressed in such a way that part of it actually extends into the cavity in the open socket 401. The pressure applied by the dressing pouch 101 substantially assists the wound site in achieving hemostasis.

Additionally, the dressing pouch 101 can be compressed against and into the exposed tooth socket 401 to fully engage the cannabinoid-infused phytotherapeutic matrix 102 with the exposed tooth socket 401. The phytotherapeutic matrix 102 substantially covers and encompasses the exposed tooth socket and proximate tissue to relieve pain and reduce inflammation, while fitting substantially between contiguous teeth. The therapeutic effectiveness of the phytotherapeutic matrix 102 is significantly influenced by its proximity to the exposed tooth socket and proximate soft tissue. The proximity of the phytotherapeutic matrix 102 to the exposed tooth socket is directly affected by the amount and degree of compression of the dressing pouch 101 between the phytotherapeutic matrix 102 and the exposed socket.

Referring now to FIGS. 6 & 7, the dressing pouch 101 has substantially conformed to the exposed tooth socket 401. These figures illustrate how the dressing pouch 101 can compress against and into an exposed tooth socket 401 to achieve substantial proximity between the phytotherapeutic matrix 102 and the exposed tooth socket 401 and proximate soft tissue. Similarly, dressing 100 will likewise conform to the complex anatomy and topology of any iatrogenic trauma or surgical site. Accordingly, in the embodiments where the phytotherapeutic matrix 102 is thermally active, cold therapy can be applied to the exposed tooth socket 401, or surgical site, and proximate soft tissue by virtue of the proximity of the phytotherapeutic matrix 102. The dressing pouch 101 along with matrix material 102 absorbs any exudate from the wound, such as serosanguineous fluid, and provides pressure to the socket and proximate tissue to promote hemostasis. Further, as depicted in the embodiment illustrated in FIG. 7, the side arms of the dressing 100 can extend beyond the tooth line to engage both sides of the gingival tissue and oral epithelium proximate the exposed tooth socket 401. Otherwise stated, the side arms of the dressing 100 can extend down below the tooth line and flank both sides of the exposed tooth socket to engage proximate gum tissue, thereby applying therapy to the blood vessels in the gum tissue below the extraction site. This helps relieve pain and decrease inflammation associated with the exposed tooth socket.

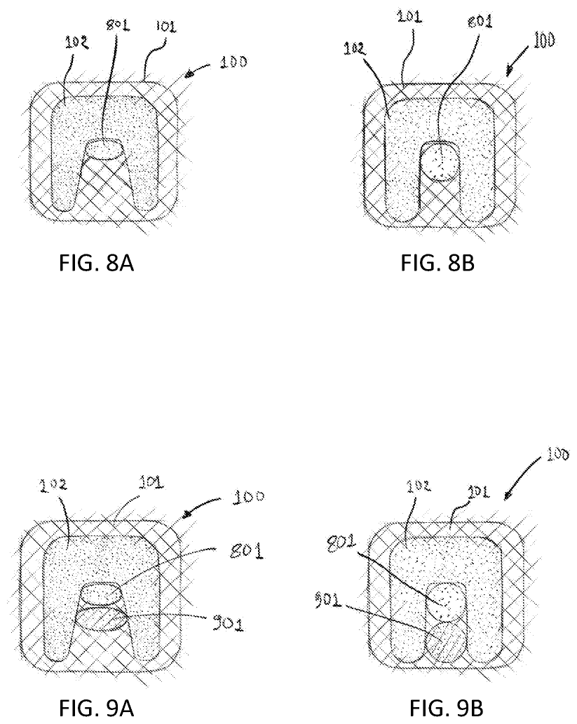

FIGS. 8-9B depict example embodiments of the phytotherapeutic matrix 102, in accordance with various embodiments of the present general inventive concept. More specifically, FIG. 8A depicts an example embodiment with the phytotherapeutic matrix 102 having tapered and angled side arms. A moisture-liberating element 801 is also included, embedded and contained substantially within the dressing pouch 101. FIG. 8B depicts an example embodiment with the phytotherapeutic matrix having side arms disposed substantially perpendicularly from the socket cover portion. FIG. 8B also includes a moisture-liberating element 801 embedded and contained substantially within the dressing pouch 101. FIGS. 9A & 9B depict both example embodiment phytotherapeutic matrix 102 discussed above, but further includes a topical agent element 901 also embedded and contained substantially within the dressing pouch 101.

In the embodiments illustrated in FIGS. 8A & 8B, moisture-liberating elements 801 are embedded and contained substantially within the dressing pouch 101, substantially adjacent to the phytotherapeutic matrix 102. In the illustrated embodiments, the moisture-liberating element 801 is comprised of hydrogel. It will be understood that hydrogel is comprised of a network of hydrophilic polymer chains. Over time, the hydrogel elements 801 in the illustrated embodiments release moisture to the exposed tooth socket and proximate tissue. It is generally accepted that keeping the exposed sockets moist and clean may help prevent alveolar osteitis, or dry sockets. Although the oral cavity is substantially moist already, the dressing pouch 101 and matrix material 102 can have a tendency to cause dryness by absorbing a significant portion of the proximate fluid, thereby depriving the wound site and proximate tissue of moisture. Incorporating a hydrogel element 801 can help ensure that the exposed tooth socket remains moist. Similarly, other moisture-liberating elements 801 containing hydrocolloids, hydrofibres, and/or any combination thereof can also be used to achieve similar results without departing from the scope or spirit of the present general inventive concept.

In the embodiments depicted in FIGS. 9A & 9B, a topical agent element 901 is included. The topical agent elements 901 can be comprised of myriad different substances that can be applied topically to the wound site to promote healing and/or relieve pain and inflammation. For example, cannabinoid-infused clove oil (eugenol) can be included in the element 901 to provide pain relief and prevent infection. In some embodiments, medications like benzocaine, iodophorm, soluble aspirin, zinc oxide, or any combination thereof, can be included in the topical agent element 901. Topical agent elements 901 containing analgesics, such as eugenol, can be particularly effective immediately following a surgical procedure and in particular tooth extraction when pain is almost always present. Analgesics and other medications are also effective in cases when dry sockets form after tooth extraction. One skilled in the art will recognize that other topical substances can be included without departing from the scope or spirit of the present general inventive concept.

In the illustrated embodiments, the moisture-liberating elements 801 are disposed between the topical agent elements 901 and the phytotherapeutic matrix 102, all embedded and contained substantially within the dressing pouch 101. Most topical agents envisioned to be included in the elements 901 are water soluble. Therefore, disposing the moisture-liberating element 801 between the topical agent and the phytotherapeutic matrix 102 will ensure that the topical agent element 901 becomes moist, thus facilitating topical application to the surgical site or exposed tooth socket 401. Furthermore, the therapeutic effectiveness of the topical agent element 901 is optimized by disposing it proximate to the wound site. However, one skilled in the art will understand that the relative disposition of the moisture-liberating elements 801 and the topical agent elements 901 described herein is non-limiting.

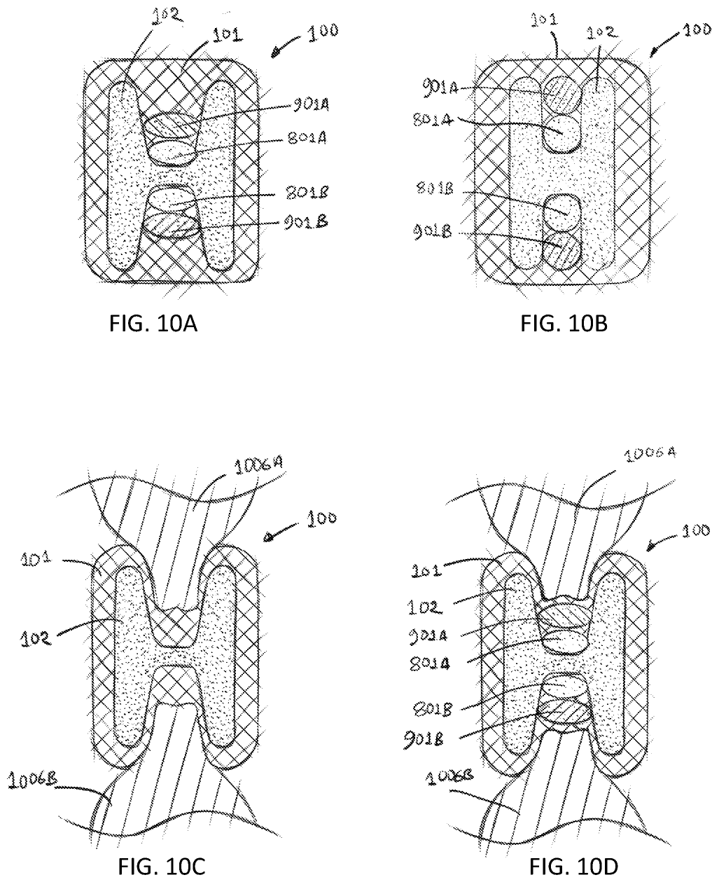

In the embodiments illustrated in FIGS. 10A-10D, the phytotherapeutic matrix 102 is configured, generally, as an “H.” FIG. 10A depicts an example embodiment of matrix 102 with tapered and angled side arms, whereas FIG. 10B depicts another example embodiment phytotherapeutic matrix 102 with non-tapered, substantially perpendicular arm members. Both of the example embodiments illustrated in FIGS. 10A & 10B include hydrogel elements 801A & 801B and topical agent elements 901A & 901B. These embodiments accommodate substantially aligned, maxillary and mandibular iatrogenic trauma particularly including exposed tooth sockets. Otherwise stated, the present embodiments can provide therapy following an extraction of substantially aligned mandibular and maxillary teeth. For example, oral, hockey injuries frequently effectuate or necessitate extraction of substantially aligned maxillary and mandibular teeth. The present embodiments provide a therapeutic option for those injuries, in particular, contained within a single wound dressing 100.

FIGS. 10C & 10D portray cross-sectional views of example embodiment, “H-shaped” dental wound dressings 100 engaging substantially aligned maxillary and mandibular exposed tooth sockets. The example embodiment depicted in FIG. 10C includes only a phytotherapeutic matrix 102 embedded and contained substantially within a dressing pouch 101, whereas the example embodiment in FIG. 10D includes a phytotherapeutic matrix 102, hydrogel elements 801A & 801B, and topical agent elements 901A & 901B embedded and contained substantially within a dressing pouch 101. Proximate gum tissue is indicated at 1006A & 1006B. As shown, the side arms of the phytotherapeutic matrix 102 flank the lingual and facial surfaces of the proximate gum tissue 1006A & 1006B and provide therapy to the blood vessels contained therein.

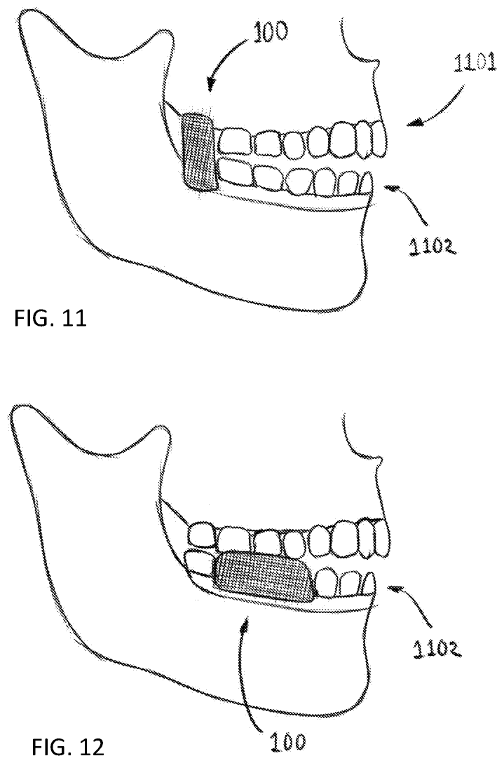

FIGS. 11-14 depict example embodiment dental wound dressings 100 that engage an extensive surgical site, for example, a plurality of exposed tooth sockets. For instance, FIG. 11 depicts an example embodiment wound dressing 100 engaging substantially aligned maxillary 1101 and mandibular 1102 tooth sockets. More specifically, the dental dressing 100 in the illustrated embodiment is engaging substantially aligned and exposed third molar, or wisdom tooth, sockets. This embodiment accommodates the “H-shaped” example embodiment phytotherapeutic matrix 102 depicted in FIGS. 10A & 10B. The presently depicted embodiment is installed within a patient's oral cavity by placing the dressing 100 in the desired location while the oral cavity is fully extended or open. The patient will then close the oral cavity to the extent permitted by the dressing 100 to compress the dressing pouch 101 against the exposed tooth sockets and proximate tissue, thereby substantially engaging the phytotherapeutic matrix 102 with the exposed sockets and proximate tissue.

FIG. 12 depicts an example embodiment wound dressing 100 engaging an extended surgical site, for example, a plurality of adjacent mandibular 1102 tooth sockets. The illustrated embodiment's phytotherapeutic matrix 102 contains the same, substantially “U” shaped cross sections portrayed in FIGS. 3A & 3B, but is elongated to cover a plurality of adjacent, exposed tooth sockets. Like the embodiment illustrated in FIG. 11, installation into a patient's oral cavity can be achieved by opening the oral cavity, inserting the dressing 100 into the selected location, and gently closing the oral cavity to compress the dressing pouch 101 and matrix 102 against and into the exposed sockets.

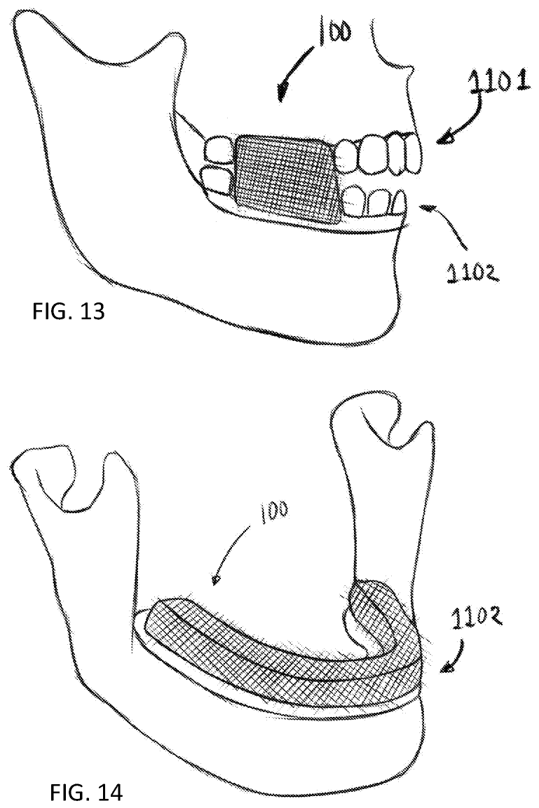

FIG. 13 depicts an example embodiment wound dressing 100 simultaneously engaging a plurality of adjacent, substantially aligned, maxillary 1101 and mandibular 1102 exposed tooth sockets. In the present embodiment, the phytotherapeutic matrix 102 contains an “H-shaped” cross section illustrated in either FIG. 10A or FIG. 10B, but is elongated to accommodate a plurality of adjacent tooth sockets.

FIG. 14 depicts an example embodiment dental wound dressing 100 engaging an entire row of exposed, mandibular 1102 tooth sockets. In this regard, the presently illustrated embodiment resembles a conventional mouthpiece. The presently illustrated embodiment can be used following the extraction of an entire row of teeth, frequently as part of the preparation for installing dentures. One skilled in the art will recognize that the present embodiment can also engage a row of exposed, maxillary 1101 tooth sockets. Thus, the present embodiment provides a therapeutic option for the pain associated with a full row tooth extraction in a single dressing 100.

In some instances, a patient has both rows of teeth extracted. Otherwise stated, it is possible to have both maxillary 1101 and mandibular 1102 full row extractions at substantially the same time. Therefore, in some embodiments of the present general inventive concept, two dental dressings 100 having “U-shaped” cross-sections can each independently engage the maxillary row 1101 and mandibular row 1102. In this embodiment, because two distinct dental dressings 100 are being used, a straw or other feeding apparatus can be inserted into the oral cavity between the two dental dressings 100. On the other hand, in other embodiments, one dental dressing having an “H-shaped” cross section can engage both the maxillary and mandibular row of exposed tooth sockets. However, in this embodiment, a straw or like device will not be able to be inserted into the patient's oral cavity because of the occluding dental dressing engaging both the upper and lower rows of tooth sockets (or surgical site).

The embodiments described herein provide a dental dressing 100 for an iatrogenic trauma or (extended) surgical site including a phytotherapeutic matrix 102 embedded and contained substantially within a dressing pouch 101. The dressing pouch 101 can be adaptable to conform to various shapes and contours as required by the particular anatomy and underlying topology. The phytotherapeutic matrix 102 can be sized and shaped to engage an exposed tooth socket and flank opposing sides of the oral epithelium, while fitting substantially between contiguous teeth. In some embodiments, the cannabinoid-infused phytotherapeutic matrix 102 includes a semi-rigid, substantially non-migrating thermally active gel-like material contained within a housing member. Following a tooth extraction, for example, patients can place the dental dressing 100 in a cold environment so that the dressing becomes cold. Subsequently, the patient can remove the dental dressing 100 from the cold environment and introduce it into their oral cavity. The dressing pouch 101 can be compressed into the exposed socket, thereby engaging the embedded phytotherapeutic matrix 102 with the exposed tooth socket. The dressing pouch 101, and the applied pressure, will help absorb exudate and promote vasoconstriction, while the cold phytotherapeutic matrix 102 will reduce inflammation, promote vasoconstriction, and relieve pain.

In other embodiments, the phytotherapeutic matrix 102 includes ammonium nitrate and water substantially contained within a polymer-based housing member. Patients/caregivers can cause the phytotherapeutic matrix 102 to become cold by effectuating an endothermic reaction with the ammonium nitrate and water, by, for example, shaking the matrix 102 or inserting water into a housing member that already contains ammonium nitrate. Similarly, after the endothermic reaction takes place to produce a cold phytotherapeutic matrix 102, the dental dressing 100 can be placed within the patient's oral cavity in the same manner described herein above.

In some embodiments of the dental dressing 100, the phytotherapeutic matrix 102 is shaped substantially like a “U,” and sized to engage, for example, one exposed tooth socket. In other embodiments, the phytotherapeutic matrix 102 is shaped generally like a “U,” but with tapered side members angularly extending from a cover portion. In yet other embodiments, the phytotherapeutic matrix 102 has a “U-shaped” cross section, but is elongated to substantially engage an extended surgical site, for example, a plurality of adjacent, exposed tooth sockets. In yet other embodiments, the phytotherapeutic matrix 102 is shaped generally like an “H,” with perpendicular or angled, tapered or non-tapered side members extending from a cover portion, to substantially engage substantially aligned maxillary 1101 and mandibular 1102 exposed tooth sockets. In other embodiments, the phytotherapeutic matrix 102 has an “H-shaped” cross section, but is elongated to substantially engage an extended surgical site, for example, a plurality of adjacent, substantially aligned maxillary and mandibular exposed tooth sockets.

In some embodiments, the dental dressing 100 includes a hydrogel element 801 embedded and contained substantially within the dressing pouch 101. In other embodiments, the dental dressing 100 includes an element 801 substantially comprised of hydrogel, hydrocolloid, hydrofibre, and/or any combination thereof. One skilled in the art will recognize that other, moisture-liberating elements can also be used without deviating from the scope or spirit of the general present inventive concept. These moisture-liberating elements 801 are particularly applicable in instances where a dry socket evolves after tooth extraction.

In some embodiments, the dental dressing 100 includes an active topical agent element 901. In other embodiments, the active topical agent element 901 includes medications like eugenol, benzocaine, iodophorm, soluble aspirin, zinc oxide, or any combination thereof. One skilled in the art will recognize that the present general inventive concept is not limited to the use of the specific substances described herein, but can also encompass other, topical agents that can be included in the element 901 for topical application to an exposed tooth socket.

Numerous variations, modifications, and additional embodiments are possible, and accordingly, all such variations, modifications, and embodiments are to be regarded as being within the spirit and scope of the present inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

The above described embodiments are set forth by way of example and are not for the purpose of limiting the scope of the present inventive concept. It will be readily apparent that obvious modifications, derivations and variations can be made to the embodiments without departing from the scope of the present inventive concept. Accordingly, the claims appended hereto should be read in their full scope including any such modifications, derivations and variations.

What is claimed is:

1. A dental dressing pouch for an oral surgical site, which comprises:
a three-dimensional absorbent matrix including a porous dressing material to assist in the removal of exudate associated with iatrogenic trauma, and;
at least one cannabinoid-infused therapeutic phytoconstituent within said porous dressing material to provide substantially localized therapy to a surgical site, wherein said at least one cannabinoid-infused therapeutic phytoconstituent contained within said porous dressing material form a pliable pouch shapeable to substantially engage an iatrogenic trauma fitting generally over and about the surgical site.

2. The dressing pouch of claim 1, wherein at least part of said three-dimensional dressing matrix substantially conforms to the surgical site, thereby engaging said at least one cannabinoid-infused therapeutic phytoconstituent intimately with the iatrogenic trauma.

3. The dressing pouch of claim 1, wherein said dressing pouch is one of a filter-paper, a muslin, a silk, a cotton gauze, a tulle, a cotton, and/or a combination thereof.

4. The dressing pouch of claim 1, wherein said dressing pouch includes one of a hydrogel, an alginate, a hydrofibre, a chitosan, and/or a combination thereof.

5. The dressing pouch of claim 1, which further includes a tea and turmeric mixed with said at least one cannabinoid within said porous dressing material.

6. The dressing pouch of claim 5, wherein said mixture is substantially non-migrating.

7. The dressing pouch of claim 6, wherein said porous dressing material is formed in a generally rectangular-shaped porous dressing pouch.

8. The dressing pouch of claim 6, wherein said porous dressing material is formed in a generally circular-shaped porous dressing pouch.

9. The dressing pouch of claim 6, wherein said porous dressing material is formed in a generally U-shaped porous dressing pouch to engage one or more exposed alveoli.

10. The dressing pouch of claim 6, wherein said porous dressing material is formed in substantially U-shaped cross section and is elongated to engage a plurality of adjacent exposed alveoli.

11. The dressing pouch of claim 6, wherein said porous dressing material is formed wherein said porous dressing pouch is substantially H-shaped to engage substantially aligned maxillary and mandibular exposed alveoli.

12. The dressing pouch of claim 6, wherein said porous dressing pouch includes a substantially H-shaped cross section and is elongated to engage a plurality of adjacent, substantially aligned, exposed maxillary and mandibular alveoli.

13. The dressing pouch of claim 1, wherein said porous dressing material further includes a moisture-liberating element.

14. The dressing pouch of claim 1, wherein said porous dressing material includes a moisture-liberating element having at least one of a hydrogel, hydrocolloid, and hydrofibre.

15. The dressing pouch of claim 1, wherein said porous dressing material includes a topical agent element.

16. The dressing pouch of claim 15, wherein said topical agent element includes clove oil (eugenol) infused with cannabidiol.

17. The dressing pouch of claim 16, wherein said topical agent element includes one of benzocaine, iodophorm, soluble aspirin, zinc oxide, and/or a combination thereof.

18. The dressing pouch of claim 1, wherein said cannabinoid-infused therapeutic phytoconstituent further includes a thermally active gel-like substance.

19. The dressing pouch of claim 15, wherein said thermally active gel-like substance provides localized cooling as a therapeutic benefit.
